SEQUENCE LISTING (2 sequences)

SEQ ID NO: 1 - DNA, 20 bp, synthetic construct
  source 1..20: mol_type = other DNA
agagtttgatcctggctcag

SEQ ID NO: 2 - DNA, 19 bp, synthetic construct
  source 1..19: mol_type = other DNA
ggttaccttgttacgactt

(19) United States
(12) Patent Application Publication
(10) Pub. No.: US 2024/0360480 A1
(43) Pub. Date: Oct. 31, 2024

(54) MARINE-DERIVED NANOSELENIUM-PRODUCING STRAIN AND METHOD FOR PREPARING NANOSELENIUM BY USING STRAIN

(71) Applicant: Dalian Minzu University, Dalian City (CN)

(72) Inventors: Chunshan Quan, Dalian City (CN); Liying Zhang, Dalian City (CN); Liming Jin, Dalian City (CN); Haodi Ma, Dalian City (CN); Xiaonan Xu, Dalian City (CN); Yan Xu, Dalian City (CN); Mingjie Ren, Dalian City (CN)

(73) Assignee: Dalian Minzu University, Dalian City (CN)

(21) Appl. No.: 18/771,445

(22) Filed: Jul. 12, 2024

Related U.S. Application Data
(63) Continuation of application No. PCT/CN2023/128041, filed on Oct. 31, 2023.

(30) Foreign Application Priority Data
Jan. 17, 2023 (CN) 202310062378.3

(51) Int. Cl. C12P 3/00; C12N 1/20; C12R 1/07

(52) U.S. Cl. CPC C12P 3/00; C12N 1/205; C12R 2001/07

(57) ABSTRACT

A marine-derived nanoselenium-producing strain and a method for preparing nanoselenium by using the strain are provided in the present disclosure, belonging to the field of nano biomaterials. According to the present disclosure, a Bacillus strain capable of preparing nanoselenium is screened from sediments taken from the Arctic Ocean, named Bacillus sp. Q72, which has been deposit in the China General Microbiological Culture Collection Center, and the deposit number is CGMCC No.26355. The extracellular fluid of this strain is used to directly reduce high concentrations of sodium selenite to obtain selenium nanoparticles.

### CROSS-REFERENCE TO RELATED APPLICATIONS

This application is a continuation of PCT/CN2023/128041, filed Oct. 31, 2023 and claims priority of Chinese Patent Application No. 202310062378.3, filed on Jan. 17, 2023, the entire contents of which are incorporated herein by reference.

### INCORPORATION BY REFERENCE STATEMENT

The Sequence Listing XML associated with this application is provided electronically in XML file format and is hereby incorporated by reference into the specification. The name of the XML file containing the Sequence Listing is “US2024-6808.xml”. The XML file is 2,722 bytes, created on Jul. 10, 2024, and is being submitted electronically via USPTO Patent Center.

### TECHNICAL FIELD

The present disclosure relates to the field of nano biomaterials, and in particular to a marine-derived nanoselenium-producing strain and a method for preparing nanoselenium by using the strain.

### BACKGROUND

Selenium is an essential trace element for the human body. It has been found that selenium may participate in the regulation of redox balance in the human body by acting as an active centre for antioxidant enzymes such as TrxR (thioredoxin reductase), GSH (glutathione), GPx (peroxidase) and DIO (iodothyronine deiodinase). The main forms of selenium are inorganic selenium (selenates, selenites), organic selenium (selenoproteins, selenopolysaccharides, etc.) and monomeric selenium. With the rapid development of nanotechnology, selenium nanoparticles (SeNPs) with anticancer, antibacterial and antioxidant effects, good biocompatibility and low biotoxicity have gradually attracted attention.

Nanoselenium is a kind of monolithic selenium, mostly red in colour, which is essentially inorganic selenium. However, it is a new type of selenium supplement with higher safety and absorbability owing to the nanoscale size and nano effect, which distinguishes it from traditional inorganic selenium. People mainly manufacture selenium-fortified foods by applying selenium fertiliser to crops and artificially adding selenium supplements to foods to meet their selenium needs. With low toxicity and a wide range of safe concentrations, nanoselenium is considered the substance of choice for selenium supplementation. In addition, nanoselenium is 5 times more effective than inorganic selenium and 2.5 times more effective than organic selenium in scavenging hydroxyl radicals in the human body. As a biological nutrient with multiple physiological functions, nanoselenium has been widely used in the treatment of more than 40 diseases, such as tumours, osteochondrosis, diabetes, heart disease and so on.

The methods used for the synthesis of nanoselenium include physical reduction, chemical reduction and biological reduction. Physical reduction methods mainly include ultraviolet radiation, laser ablation and hydrothermal techniques. Chemical synthesis mainly uses ascorbic acid, glucose, sulphur dioxide, sodium dodecyl sulphate and other catalytic reduction of high valence selenium to form nanoselenium. However, a series of problems such as environmental pollution, high cost, high energy consumption, and insecurity associated with physical and chemical reduction methods have limited the development and wide application of SeNPs in food, biomedical, and feed additives. In recent years, biosynthesis of nanoparticles has been widely welcomed owing to its cost-effectiveness, accessible source of raw materials, low toxicity and pharmacological potential, possessing the advantages of simplicity, safety, high biocompatibility, environmental friendliness, and recyclability, and the nature, size, and morphology of the nanoparticles may be easily controlled by varying the temperature of the reaction, the pH, the time of the reaction, the concentration of the metal ions, and the amount of the organic material. Currently, various microorganisms, such as bacteria, yeasts, and fungi, have been considered as environmentally friendly green nanofactories for the preparation of nanoselenium materials. For instance, Bacillus licheniformis cultured under sodium selenite solution induces the conversion of toxic selenite ions (SeIV) into non-toxic elemental selenium nanoparticles (Se0). Alternatively, LactoBacillus acidophilus, Streptococcus thermophilus, LactoBacillus casei, or Pseudomonas acidophilus may also synthesize nanoselenium, and the nanoselenium synthesised using microorganisms is possible to modify the toxicity of selenium and is easy to obtain the nanoparticles, thus showing great prospects for application in various fields.

However, the existing biosynthesis method of nanoselenium mostly adopts the strategy of simultaneous bacterial fermentation and synthesis of nanoselenium, and the bacterium needs to be crushed at a later stage, and then go through extraction and centrifugation to obtain nanoselenium, which is a synthesis strategy that has the drawbacks of cumbersome separation steps, low separation efficiency, and easy to deposit bacterial debris in nanoselenium. In addition, more bacterial debris adhered to the surface of nanoselenium is not easy to be removed, resulting in increased cytotoxicity.

### SUMMARY

The objective of the present disclosure is to provide a marine-derived nanoselenium-producing strain and a method for preparing nanoselenium by using the strain, so as to solve the problems existing in the prior art. Using the extracellular fluid of this strain, high concentration of sodium selenite may be directly reduced to obtain nanoselenium. This method has the advantages of high reduction efficiency and simple separation process, and the synthesised nanoselenium has uniform particle size, no impurities such as bacterial debris, and low cytotoxicity.

In order to achieve the above objectives, the present disclosure provides the following scheme.

The present disclosure provides a marine-derived nanoselenium-producing strain, where the nanoselenium-producing strain is named Bacillus sp. Q72, and is deposited in the China General Microbiological Culture Collection Center, with a deposit number of CGMCC No. 26355.

The present disclosure also provides a method for preparing nanoselenium by using the nanoselenium-producing strain, including using an extracellular fluid of the nanoselenium-producing strain for reducing sodium selenite to prepare the nanoselenium.

Optionally, the method includes following steps:
step 1, preparing the extracellular fluid of the nanoselenium-producing strain;
step 2, adding a sodium selenite solution into the extracellular fluid, and reacting for 24 hours (h); and
step 3, centrifuging a mixed solution after culture, collecting a precipitate, washing, and drying under vacuum to obtain the nanoselenium.

Optionally, in the step 2, a final concentration of the sodium selenite solution is 40 millimoles per liter (mmol/L); and reaction conditions include 37 degrees Celsius (° C.), 180 revolutions per minute (r/min) and 24 h.

Optionally, in the step 3, conditions of the centrifuging includes 10000 r/min for 20 min; and a temperature for the drying under vacuum is 60° C.

Optionally, in the step 1, a specific preparation method of the extracellular fluid is as follows:
S1, subjecting a bacterial solution of the strain of a stable growth phase to centrifugation, placing a supernatant on ice and labelling as S-1;
S2, washing a precipitate after centrifugation in the S1 by precooled ultrapure water, adding with a Tris-HCl solution for re-suspension, obtaining a supernatant after centrifugation and labelling as S-2;
S3, adding precooled sucrose solution into a precipitate after centrifugation in the S2 for re-suspension, and collecting a supernatant after centrifugation, and labelling as S-3; and
S4, combining supernatants S-1, S-2 and S-3 to obtain the extracellular fluid.

Optionally, in the S1 and the S2, conditions of the centrifugation are 8000 r/min for 15 min.

Optionally, in the S2, a concentration of the Tris-HCl solution is 10 mmol/L, with a pH of 8.0.

Optionally, in the S3, a mass concentration of the sucrose solution is 25%; and conditions of the centrifugation are 10000 r/min and 15 min.

The present disclosure also provides an application of the nanoselenium-producing strain in preparing nanoselenium.

The present disclosure has the following technical effects.

According to the present disclosure, a Bacillus strain capable of preparing nanoselenium is screened from sediments taken from the Arctic Ocean, and high-concentration sodium selenite may be directly reduced to obtain nanoselenium by using the extracellular fluid of the strain. The reduction efficiency of this method is high, and the separation process of nanoselenium obtained by reduction is simple, and the synthesized nanoselenium has uniform particle size, no impurities such as bacterial fragments, and low cytotoxicity.

### BRIEF DESCRIPTION OF THE DRAWINGS

In order to explain the embodiments of the present disclosure or the technical scheme in the prior art more clearly, the drawings needed in the embodiments are briefly introduced below. Obviously, the drawings described below are only some embodiments of the present disclosure, and other drawings may be obtained according to these drawings without creative work for ordinary people in the field.

FIG. 1 shows the results of co-culture of extracellular fluid, perimembrane fluid and intracellular fluid of Bacillus sp. Q72 with Na₂SeO₃ solution for 24 hours (h).

FIG. 2 is a graph showing the time-dependent variation of sodium selenite reduction by extracellular fluid of Bacillus sp. Q72.

FIG. 3 is a scanning electron microscope photograph of the extracellular fluid of Bacillus sp. Q72 reducing sodium selenite to produce nanoselenium.

FIG. 4 is the Zeta potential diagram of the extracellular fluid of Bacillus sp. Q72 for reducing sodium selenite to produce nanoselenium.

FIG. 5 is the X-ray Diffraction (XRD) spectrum of the extracellular fluid of Bacillus sp. Q72 for reducing sodium selenite to produce nanoselenium.

FIG. 6 shows the infrared spectrum of the extracellular fluid of Bacillus sp. Q72 for reducing sodium selenite to produce nanoselenium.

FIG. 7 shows the toxicity results of extracellular fluid of Bacillus sp. Q72 to HCoEpiC cells by reducing sodium selenite to produce nanoselenium.

FIG. 8 shows is a process illustrating the method for preparing nanoselenium by using the nanoselenium-producing strain provided by the present disclosure.

FIG. 9 shows is a process illustrating the preparation method of the extracellular fluid provided by the present disclosure.

### DETAILED DESCRIPTION OF THE EMBODIMENTS

A number of exemplary embodiments of the present disclosure are now described in detail, and this detailed description should not be considered as a limitation of the present disclosure, but should be understood as a rather detailed description of certain aspects, characteristics and embodiments of the present disclosure.

It should be understood that the terminology described in the present disclosure is only for describing specific embodiments and is not used to limit the present disclosure. In addition, for the numerical range in the present disclosure, it should be understood that each intermediate value between the upper limit and the lower limit of the range is also specifically disclosed. The intermediate value within any stated value or stated range and every smaller range between any other stated value or intermediate value within the stated range are also included in the present disclosure. The upper and lower limits of these smaller ranges can be independently included or excluded from the range.

Unless otherwise specified, all technical and scientific terms used herein have the same meaning as commonly understood by one of ordinary skill in the art to which this present disclosure relates. Although the present disclosure only describes the preferred methods and materials, any methods and materials similar or equivalent to those described herein may also be used in the practice or testing of the present disclosure. All documents mentioned in this specification are incorporated by reference to disclose and describe methods and/or materials related to the documents. In case of conflict with any incorporated document, the contents of this specification shall prevail.

It is obvious to those skilled in the art that many improvements and changes may be made to the specific embodiments of the present disclosure without departing from the scope or spirit of the present disclosure. Other embodiments will be apparent to the skilled person from the description of the present disclosure. The specification and embodiments of this application are only exemplary.

The terms “including”, “comprising”, “having” and “containing” used in this specification are all open terms, which means including but not limited to.

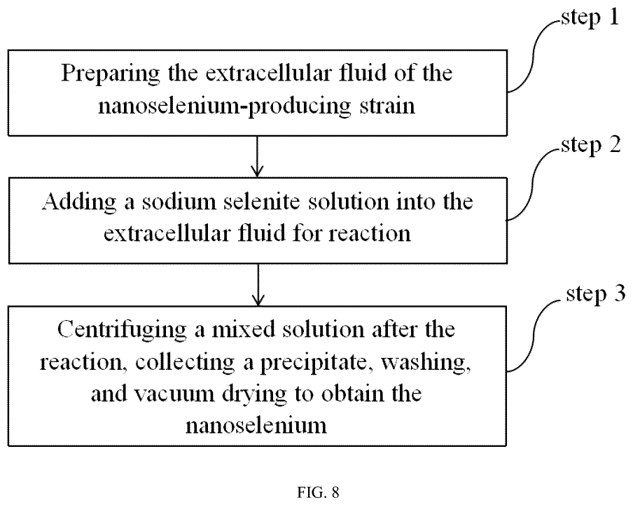

The present disclosure provides a method for preparing nanoselenium by using a nanoselenium-producing strain, including using an extracellular fluid of the nanoselenium-producing strain for reducing sodium selenite to prepare the nanoselenium, and the method includes the following steps as shown in FIG. 8:
step 1, preparing the extracellular fluid of the nanoselenium-producing strain;
step 2, adding a sodium selenite solution into the extracellular fluid for reaction; and
step 3, centrifuging a mixed solution after the reaction, collecting a precipitate, washing, and drying under vacuum to obtain the nanoselenium.

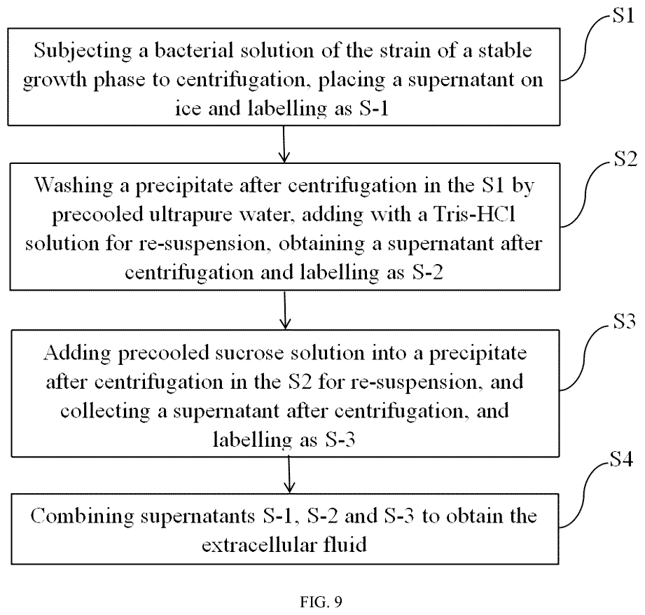

According to the present disclosure, a preparation method of the extracellular fluid includes following steps as shown in FIG. 9:
S1, subjecting a bacterial solution of the strain of a stable growth phase to centrifugation, placing a supernatant on ice and labelling as S-1;
S2, washing a precipitate after centrifugation in the S1 by precooled ultrapure water, adding with a Tris-HCl solution for re-suspension, obtaining a supernatant after centrifugation and labelling as S-2;
S3, adding precooled sucrose solution into a precipitate after centrifugation in the S2 for re-suspension, and collecting a supernatant after centrifugation, and labelling as S-3; and
S4, combining supernatants S-1, S-2 and S-3 to obtain the extracellular fluid.

### Embodiment 1 Screening and Identification of Strains

### 1. Isolation and Screening of Strains

In the early stage, the inventor isolated 40 strains from the marine sediments of the Arctic Ocean and kept them frozen at −80 degrees Celsius (° C.).

### (1) Activation of Marine Strains

The 40 strains frozen at −80° C. are taken out and put into the ice box for thawing, the ultraviolet of the ultra-clean workbench is turned off, the alcohol lamp is lit, the inoculation ring is burnt red, and then cooled down, near the alcohol lamp, the appropriate amount of bacteria solution is dipped, and the LB solid plate medium is used for scribing inoculation, and then the plate is inverted, and incubated in the thermostat box for 16 h. After the colonies are grown out, a single colony is picked out, and inoculated into 10 mL LB medium, and it is placed in a constant temperature shaker and incubated at 33° C. and 180 r/min for 24 h for activation.

### (2) Tolerance Test of the Strains

150 microliters (μL) of activated bacteria solution is added to 5 milliliters (mL) of LB medium containing 5 millimoles per liter (mmol/L) selenite solution, and cultured for 24 h at 33° C. and 180 r/min, and the OD value of the strain is determined. Six strains with good growth are selected from 40 marine strains for tolerance test.

150 μL of the activated bacteria solution is put in 5 mL of LB medium, and then selenite solutions with different solubility are added to make the final concentration of the system 10, 20, 40, 60, 80, 100, 200, 300, 400 and 500 mmol/L respectively, followed by mixing well, placing in a constant temperature shaker and culturing at 33° C. and 180 r/min for 24 h; and the reduction of sodium selenite with different concentrations by each strain is observed. A strain with the strongest tolerance to sodium selenite is screened out from 6 strains and stored at −80° C.

### 2. Identification of Strains

### (1) Obtaining of a Single Colony by a Plate Scribing Method

The selected strain is taken out from the refrigerator at −80° C., placed in the foam box with crushed ice to be thawed and mixed well, and the bacteria solution is sucked into the LB liquid medium, and 3 test tubes for each strain are activated, and placed in the constant temperature shaker at 37° C. to be activated and cultivated for 16 h. In the ultraclean bench, an alcohol lamp is ignited, and the inoculation ring is sterilized by cauterization, and then dipped in the bacteria solution after cooling, then it is used to evenly scribe the bacteria solution onto the LB solid medium, which is placed in the biochemical culture incubator at constant temperature of 37° C. to be cultivated for 36 h. Three groups of replicates are set up

### (2) Molecular Biological Identification of the Strain

The plate that has grown with single colonies in step (1) is taken out, and the well-grown single colonies are picked up and placed in a centrifuge tube containing 150 μL of sterile water, boiled at 100° C. for 10 min, centrifuged for 2 min (12000 r/min), and the supernatant is preserved for spare use.

The supernatant is used as a template for PCR amplification using bacterial universal primers 8F and 1492r. The amplification reaction system is as follows: upstream and downstream primers are 2 μL, 10× buffer 2 μL, Taq enzyme 25 μL, deionized water 20 μL, and supernatant extract 1 μL, in which the upstream primer is 8F: 5′-AGAGTTTGATCCTGGCTCAG-3′ (SEQ ID NO: 1) and the downstream primer is 1492R: 5′-GGTTACCTTGTTACGACTT-3′ (SEQ ID NO:2); and the amplification conditions include: reaction at 95° C. for 5 min; (30× start) reaction at 94° C. for 30 s; reaction at 50° C. for 30 s; reaction at 72° C. for 1 min (30× end); reaction at 72° C. for 10 min; and storing at 4° C.

The amplified products are recovered by 1% agarose gel electrophoresis and sent to Sangon Biotech (Shanghai) Co., Ltd. for DNA detection.

The strain is identified as Bacillus sp.Q72, and is deposited in China General Microbiological Culture Collection Center (CGMCC for short; address: No. 3, No. 1 Courtyard, Beichen West Road, Chaoyang District, Beijing, with postal code of 100101) and deposit number of CGMCC No. 26355.

### Embodiment 2 Preparation of Nanoselenium by Bacillus sp.Q72

### 1. Culture of Bacillus sp.q72

### (1) Strain Activation

All the bacteria used in the experiment are cultured in LB seawater culture medium. The specific preparation method of LB seawater culture medium includes: weighing 10 grams (g) peptone, 5 g yeast extract powder and 10 g NaCl, adding 1000 mL membrane-filtered aged seawater (filtered by 0.2 micrometer (μm) filter membrane) and fully stirring and mixing in a 2 L beaker. The culture medium is sterilized (121° C., 20 min) before use.

The test tube containing 5 mL LB seawater culture medium is added with 500 μL of Bacillus sp.Q72 bacteria solution (obtained by incubating Bacillus sp. Q72 single colonies in 10 mL LB medium at 33° C. for 24 h at 180 r/min on a shaker), and cultured at constant temperature for 24 h (37° C. and 180 r/min).

### (2) Bacteria Culture

With an inoculation amount of 3% (V/V), the activated Bacillus sp. Q72 is added to 1000 mL LB seawater culture medium and cultured at constant temperature for 48 h (37° C., 180 r/min) to obtain Bacillus sp. Q72 bacteria solution in a stable growth period.

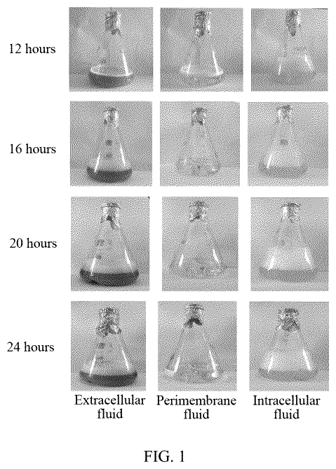

2. Determination of the Position of Bacillus sp. Q72 in Reducing Sodium Selenite to Produce Nanoselenium
(1) Taking 100 mL of Bacillus sp. Q72 bacteria solution in the stable growth period, and centrifuging for 15 min (rotating speed of 8000 r/min);
(2) taking the supernatant and storing in ice for later use, and recording as S-1; washing the precipitate with 4 mL of precooled ultrapure water, adding 100 mL 10 mM Tris-HCl (pH=8.0) for re-suspension, and centrifuging for 15 min (8000 r/min);
(3) taking the supernatant and storing for later use, and recording as S-2; adding 100 mL of 25 wt % sucrose solution (precooled) to resuspend the precipitate, and centrifuging (10000 r/min, 15 min);
(4) taking the supernatant and storing for later use, and recording as S-3; washing the precipitate with ultrapure water, then resuspending with 10 mL ultrapure water and centrifuging (10000 r/min, 15 min);
(5) taking the supernatant and storing for later use, and recording as S-4; adding 10 mL Tris-HCl (pH=7.5) solution to the precipitate, resuspending, ultrasonically crushing in ice bath (working time is 5 seconds, working interval is 3 seconds) for 20 min, and then centrifuging (10000 r/min, 15 min);
(6) storing the supernatant for later use, and recording as S-5; finally, combining the supernatants S-1, S-2 and S-3 and recording as the extracellular fluid, the S-4 as the perimembrane fluid, and the S-5 as the intracellular fluid; and
(7) respectively adding 100 mL of the extracellular fluid, the perimembrane fluid and the intracellular fluid into three-necked bottles, adding with Na₂SeO₃ solution (the final concentration is 40 mmol/L), and then culturing for 24 h at 37° C. and 180 r/min; taking samples and photos at 12 h, 16 h, 20 h, and 24 h of incubation, respectively, with the results as shown in FIG. 1.

As observed in FIG. 1 after 24 h of incubation, the extracellular fluid of strain Bacillus sp. Q72 is already changing to red colour at 12 h, indicating that nanoselenium is produced, and the system has been keeping gray colour with the extension of time. While the perimembrane and intracellular extracts show no colour change, indicating that sodium selenite is reduced to nanoselenium in the extracellular fluid of the bacterium.

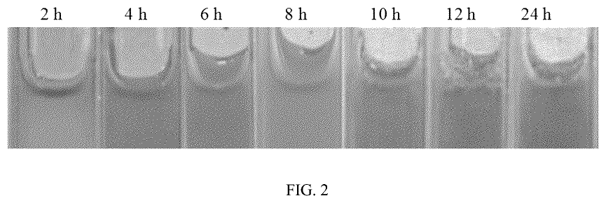

3. Bacillus sp. Q72 Reducing Sodium Selenite to Produce Nanoselenium
(1) Taking 100 mL of Bacillus sp. Q72 bacteria solution in the stable growth period, and centrifuging for 15 min (rotating speed of 8000 r/min);
(2) taking the supernatant and placing on ice for later use, and recording as S-1; washing the precipitate with 4 mL of precooled ultrapure water, adding 100 mL 10 mM Tris-HCl (pH=8.0) for re-suspension, and centrifuging for 15 min (8000 r/min);
(3) storing the supernatant for later use, and recording as S-2; adding 100 mL of 25 wt % sucrose solution (precooled) to resuspend the precipitate, and centrifuging (10000 r/min, 15 min);
(4) storing the supernatant for later use, and recording it as S-3; combining the supernatants S-1, S-2 and S-3 and recording as the extracellular fluid; and
(5) adding Na₂SeO₃ solution (final concentration of 40 mmol/L) into a three-necked bottle containing 200 mL of the extracellular fluid, and reacting for 24 h at 37° C. and 180 r/min; during the reaction, taking samples and photos at 2 h, 4 h, 6 h, 8 h, 10 h, 12 h and 24 h respectively, with results as shown in FIG. 2. As shown in FIG. 2, after co-culturing the extracellular fluid of strain Bacillus sp. Q72 with sodium selenite for 4 h, there is both red nanoselenium production and a faster rate of extracellular fluid reduction.

Subsequently, the mixed solution cultured for 24 hours is centrifuged at 10000 r/min for 20 min, and washed and precipitated with 12 mL ultrapure water and 12 mL ethanol for three times, and dried in vacuum at 60° C. for 6 h to obtain red SeNPs, which is subsequently characterized.

4. Characterization of Strain Bacillus sp. Q72 Reducing Sodium Selenite to Produce Nanoselenium

### (1) Nano Particle Size Analyzer

In order to determine the particle diameter of biosynthetic nanoselenium particles, the dried red SeNPs particles are diluted in ultra-pure water. The size and Zeta potential of lipid nanoparticles are determined by dynamic light scattering method and SZ-100 nanometer particle size analyzer.

### (2) Scanning Electron Microscope

A small amount of red SeNPs powder is taken and dissolved in anhydrous ethanol solution, the resulting nanoselenium solution is ultrasonicated, a small amount of solution is aspirated on a clean and dry silicon wafer, and when the anhydrous ethanol is completely volatilised, it is fixed by electroglue and undergoes the gold spraying process.

### (3) X-Ray Diffractometer

The red SeNPs powder is placed in the sample tank of a glass slide, and the surface is tabletted. Then it is placed in an X-ray diffractometer for testing, with a scanning angle of 10-80° and a scanning speed of 5°/min, and the nanoselenium is subjected to material analyses.

### (4) Fourier Infrared Spectrum

Red SeNPs powder is mixed with potassium bromide and tabletted, and the absorption spectrum is measured by Fourier Transform Infrared Spectroscopy (FTIR) at room temperature in the range of 400-4000 cm⁻¹, and the scanning speed is 5 kHz.

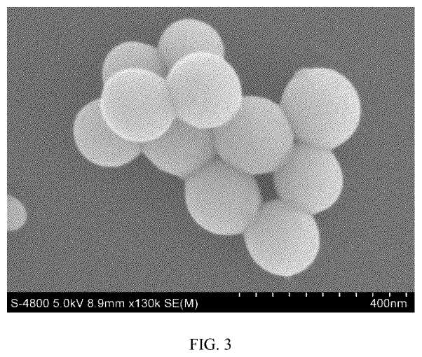

The scanning electron microscope image of nanoselenium is shown in FIG. 3. From FIG. 3, it is observed that the nanoselenium obtained by reducing sodium selenite with extracellular fluid of Bacillus sp. Q72 is spherical, with a particle size of 150-200 nm.

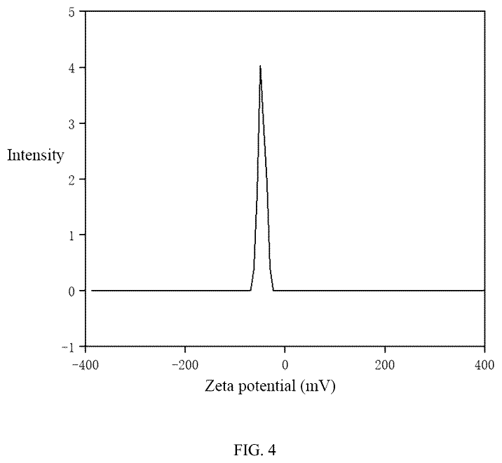

The Zeta potential diagram of nanoselenium is shown in FIG. 4, from which it may be seen that the Zeta potential value of nanoselenium is −50.1 millivolt (mV), indicating that the nanoselenium obtained by reducing sodium selenite with extracellular fluid of Bacillus sp. Q72 has high stability.

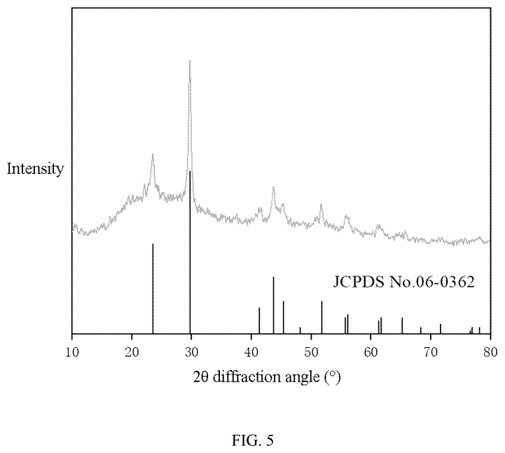

The X-ray Diffraction (XRD) spectrum of nanoselenium is shown in FIG. 5, from which it may be seen that nanoselenium originated from Bacillus sp. Q72 has a strong emission peak between 20° and 30°. Compared with the hexagonal standard card of nanoselenium (JCPDS No. 06-0362), 2θ has obvious diffraction peaks at 23.44°, 29.66°, 41.26°, 43.66°, 51.56°, 55.66° and 61.12°, which correspond to the (100), (101), (110), (102), (111), (201), and (202) crystal planes of the hexagonal crystal structure. From the XRD pattern, it may be clearly seen that the diffraction peak intensity is the highest at 29.66° corresponding to the (101) plane, which shows that the (101) plane is dominant in the nanoselenium crystal particles synthesized by reducing sodium selenite by the extracellular fluid of Bacillus sp. Q72.

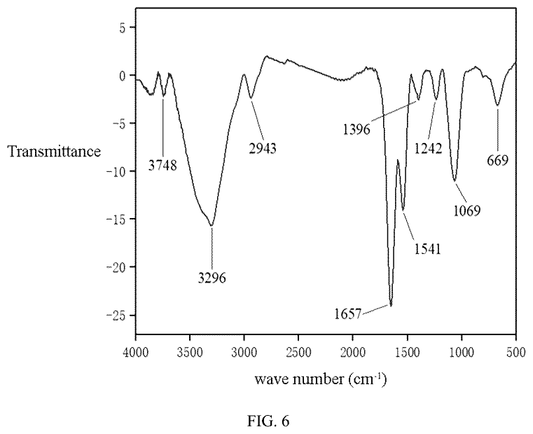

The infrared spectrum of nanoselenium is shown in FIG. 6. From FIG. 6, it is observed from the FTIR spectra that there are obvious characteristic absorption peaks at the positions of 3748^{cm-1}, 3296^{cm-1}, 2943^{cm-1}, 1657^{cm-1}, 1541^{cm-1}, 1396^{cm-1}, 1242^{cm-1}, 1069^{cm-1} and 669^{cm-1}; among them, the characteristic peak at the position of 3748^{cm-1} may be the presence of free hydroxyl group of ethanol, the peak at 3296^{cm-1} is an O—H stretching or secondary amine NH stretching vibration of the ethanol-related group; the absorption peak at 2943^{cm-1} may be the result of C—H stretching vibrations in the aliphatic group; the absorption peak at 1657^{cm-1} may be due to the presence of C═O (amide I band) stretching vibration in the amide group; the absorption peak at 1541^{cm-1} may be caused by the C═O stretching of protein amide II or the bending of N—H plane; the absorption peak at 1396^{cm-1} may be caused by C—H deformation vibration; the absorption peak at 1242^{cm-1} may be caused by amide III/CH₂ rocking vibration; the absorption peak of 1069^{cm-1} is mainly caused by C—H stretching vibration; the O—H translational vibration makes the absorption peak appear at 669^{cm-1}. The FTIR results show that there are organic substances such as lipids, protein and carbohydrates on the surface of SeNPs obtained by reducing sodium selenite with extracellular fluid of Bacillus sp. Q72. The experimental results show that the surface of nanoselenium synthesized by bacteria is attached with hydroxyl and amino groups, which shows that biomolecules containing these functional groups play an important role in the synthesis of SeNPs.

4. Cytotoxicity of Bacillus sp. Q72 in Reducing Sodium Selenite to Produce Nanoselenium

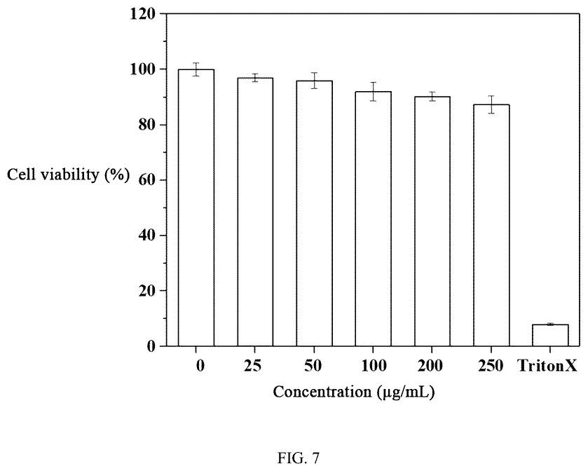

After being digested by trypsin, HCoEpiC cells in logarithmic growth phase are counted by cell counting plate, and inoculated in 96-well flat-bottom culture plate with 100 μL/well (the number of cells per well is 1.0×10⁴), with 6 repetitive wells in each group. After inoculating cells, each group is placed in an incubator with 37° C. and 5% CO₂ saturation humidity for 12 h in sterile culture; the original culture medium is removed and replaced with SeNPs aqueous solution (set the concentration gradient as 0 μg/mL, 25 μg/mL, 50 μg/mL, 100 μg/mL, 200 μg/mL, 250 μg/ml), and after adding samples, the culture is continued for 24 h, and the supernatant is sucked off 4 h before the end of the experiment; each well is added with 100 μL of 5 mg/mL MTT, incubated at 37° C. for 4 h, the supernatant is well aspirated, 100 μL of dimethylsulfoxide is added, and oscillated for 10 min, the precipitate is dissolved completely, and then the absorbance value is read at the wavelength of 490 nm with an automatic enzyme-calibrated colorimeter (A490), and the results are shown in FIG. 7. As shown in FIG. 7, the viability of HCoEpiC cells remains above 85% at a concentration of 250 ug/mL of SeNPs, indicating that the SeNPs obtained from the reduction of sodium selenite by the extracellular fluid of Bacillus sp. Q72 have low cytotoxicity.

The above-mentioned embodiments only describe the preferred mode of the present disclosure, and do not limit the scope of the present disclosure. Under the premise of not departing from the design spirit of the present disclosure, various modifications and improvements made by ordinary technicians in the field to the technical scheme of the present disclosure shall fall within the protection scope determined by the claims of the present disclosure.

What is claimed is:

1. A marine-derived nanoselenium-producing strain, wherein the nanoselenium-producing strain is named Bacillus sp. Q72, and is deposited in China General Microbiological Culture Collection Center with a deposit number of CGMCC No. 26355.

2. A method for preparing nanoselenium by using the nanoselenium-producing strain according to claim 1, comprising using an extracellular fluid of the nanoselenium-producing strain for reducing sodium selenite to prepare the nanoselenium.

3. The method according to claim 2, wherein following steps are comprised:
step 1, preparing the extracellular fluid of the nanoselenium-producing strain;
step 2, adding a sodium selenite solution into the extracellular fluid for reaction; and
step 3, centrifuging a mixed solution after the reaction, collecting a precipitate, washing, and drying under vacuum to obtain the nanoselenium.

4. The method according to claim 3, wherein in the step 1, a preparation method of the extracellular fluid comprises:
S1, subjecting a bacterial solution of the strain of a stable growth phase to centrifugation, placing a supernatant on ice and labelling as S-1;
S2, washing a precipitate after the centrifugation in the S1 by precooled ultrapure water, adding with a Tris-HCl solution for re-suspension, collecting a supernatant after centrifugation and labelling as S-2;
S3, adding precooled sucrose solution into a precipitate after the centrifugation in the S2 for re-suspension, and collecting a supernatant after centrifugation, and labelling as S-3; and
S4, combining supernatants S-1, S-2 and S-3 to obtain the extracellular fluid.

5. The method according to claim 4, wherein in the S1 and the S2, conditions of the centrifugation are 8000 revolutions per minute and 15 minutes.

6. The method according to claim 4, wherein in the S2, a concentration of the Tris-HCl solution is 10 millimoles per liter, and a pH is 8.0.

7. The method according to claim 4, wherein in the S3, a mass concentration of the sucrose solution is 25%; and conditions of the centrifugation are 10000 revolutions per minute and 15 minutes.

8. The method according to claim 3, wherein in the step 2, a final concentration of the sodium selenite solution is 40 millimoles per liter; and reaction conditions are 37 degrees Celsius, 180 revolutions per minute and 24 hours.

9. The method according to claim 3, wherein in the step 3, conditions of the centrifugation are 10000 revolutions per minute for 20 minutes; and a temperature of the drying under vacuum is 60 degrees Celsius.

10. An application of the nanoselenium-producing strain according to claim 1 in preparing nanoselenium.
